(19) United States
(12) Patent Application Publication
(10) Pub. No.: US 2024/0261477 A1
(43) Pub. Date: Aug. 8, 2024

(54) Breast fluid collection pad, method of use and kit

(71) Applicant: Dom L. Gatto, Branford, CT (US)

(72) Inventor: Dom L. Gatto, Branford, CT (US)

(21) Appl. No.: 18/445,342

(22) Filed: Jul. 26, 2023

Related U.S. Application Data
(60) Provisional application No. 63/576,302, filed on Feb. 3, 2023.

(51) Int. Cl. A61M 1/06

(52) U.S. Cl. CPC A61M 1/062

(57) ABSTRACT

A breast fluid collection pad assembly comprising: a pad having a negative capillary pressure, a cover mounted on said foam pad, said cover being provided with an adhesive coating allowing it to be mounted to the skin of a patient holding said pad over a breast and fluid indication means positioned on said cover showing the capture of breast fluids by said foam pad, said covering being provided with a target symbol for aligning the center of said cover over a breast nipple when mounted to the breast.

### RELATED APPLICATIONS

Provisional patent application Ser. No. 63/576,302 filed Feb. 3, 2023

### STATEMENT REGARDING FEDERALLY SPONSORED RESEARCH OR DEVELOPMENT

None

### REFERENCE TO SEQUENCE LISTING, A TABLE OR A COMPUTER PROGRAM LISTING COMPACT DISC APPENDIX

None.

### BACKGROUND OF THE INVENTION

### 1. Field of Invention

The field of this invention relates to a method, apparatus and kit for obtaining breast fluid obtained from the mammary ducts of women and testing the breast fluid markers contained therein. The breast fluid markers are collected on a pad for evaluating and diagnosing the markers collected on the collection pad to detect risk assessment of cancer, breast cancer, breast pre-cancer and cancer susceptibility in women.

### 2. Background of the Invention

Breast cancer is the most common form of cancer in women and is the second leading cause of cancer death in humans. Despite the many advances in diagnosing and treating breast cancer, the prevalence of this disease has been steadily rising about 1% per year since 1940. The likelihood of death due to breast cancer has remained relatively unchanged at about 27 deaths per 100,000 women. For several decades the medical community dedicated to studying breast cancer have experimented and shown that the cytological analysis of cells and other markers retrieved from the nipple aspirate fluid discharged from breast milk ducts can provide valuable information which can be used to identify patients at risk for breast cancer and patients having breast cancer. The methods which are presently used are time intensive and costly and require trained medical workers to collect the nipple aspirate fluid.

Cancer is believed to originate in the lining of breast milk ducts in the breast and human breasts are formed with 6 to 9 of these milk ducts. It is known in the prior art to use foamed polymer sponges including polyvinyl acetal sponges as collectors of fluids from patients. U.S. Pat. No. 4,098,728 issued Jul. 4, 1978 discloses the use of polyvinyl acetal material having a fast wicking and high liquid holding capacity for medical usage. U.S. Pat. No. 5,744,150 issued on Apr. 28, 1991 and U.S. Pat. No. 5,928,665 issued Jul. 27, 1999 discloses a method for producing an antimicrobial iodine. polyvinyl acetal sponge which is soaked in an aqueous bath of 20% to 70% triethylene glycol.

U.S. Pat. No. 6,613,347 issued Sep. 2, 2003 is directed toward a polyvinyl acetal sponge with a smooth outer durometer silicone skin having less porosity then the foam center. The composite wound dressing allows moisture adsorption through the skin into the PVA sponge body while presenting an outer surface precluding wound growth into the sponge material.

Patent Application PCT/USS2012/022754 claiming priority from U.S. Provisional Patent Application No. 61/441,452 filed Feb. 10, 2011 is directed towards analyzing a panel of pre-collected breast nipple discharge fluids which are tested for specific bio-markers to arrive at a prediction of breast cancer using breast nipple aspirate fluid or spontaneous discharge fluid. The quantification of specific biological markers in patients is an attractive alternative in that it is cheaper, faster, safer and provides greater specificity and sensitivity than invasive methods.

Nipple aspiration of breast fluid is cost-effective and nipple aspirate markers give detection of in situ and invasive breast cancer. Nipple aspirate fluid (NAF) can be obtained from up to 95% of women.

The teachings of the above noted patents do not aid in the resolution of a number of practical difficulties of testing for cancer or pre-cancerous conditions that are resolved by the present invention.

### SUMMARY OF THE INVENTION

The present invention is directed towards a collection pad for collecting breast fluid and the markers contained therein for testing of two or more of the bio-markers that can be identified in the breast fluid.

It is another object of the invention to provide a fluid collection pad which exerts a negative pressure on the breast and pulls breast fluid from the subject breast into the collection matrix of the breast pad for use in testing for cancer or pre-cancerous conditions of a patient.

It another object of the present invention to provide a kit which allows collection of the breast fluid with the respective markers being preserved and sent to a testing center for testing of the various markers to ascertain the present of cancer or propensity of breast cancer in the subject.

In accordance with a yet another object of the present invention there is provided a collection device for gathering and holding breast fluid collected from a patient. A composite collection pad is secured to a human breast and placed over the nipple of the breast. The pad may be used to collect normal fluid secretion into the pad through negative pressure applied by the collector pad or by applying additional suction to the breast either before the pad is used to initiate the generation of fluid or while the pad is being used. This may be accomplished by the polymeric pad which applies negative pressure or suction to the breast area during a period of non-lactation to generate breast fluid or using additional suction which is applied to the breast by mechanical devices as shown in FIGS. 5-8.

Further features and advantages of the present invention will become apparent from the detailed description of the preferred embodiment when considered together with the attached drawings and claims.

### BRIEF DESCRIPTION OF THE DRAWINGS

The present invention will be described with reference to the appended Figures, in which:

FIG. 1 shows a top plan view of the collection pad in assembled form;

FIG. 2 shows a an exploded perspective of the collection pad invention removed from the breast and nipple with the individual elements of the device separated;

FIG. 3 is a perspective view partially in phantom of the collection pad removed and placed in a shipping container or pouch for transport of the collection pad to a testing facility;

FIG. 4 is a schematic drawing of the collection pad kit and components therein.

FIG. 5 is a side elevational view showing negative pressure being applied by a flexible hollow bulb generating an outside negative pressure which can be used in the kit of FIG. 4;

FIG. 6 is an exploded view showing a syringe used in place of the hollow bulb of FIG. 5 which is used to apply constant or pulsating vacuum to the breast;

FIG. 7 is a perspective view of a battery powered vacuum device which can generate constant and pulse modes with variable psi which can be used in the kit of FIG. 4; and

FIG. 8 is a perspective view of a manual suction cup which can be used in the kit of FIG. 4.

These and other objects, advantages, and novel features of the present invention will become apparent when considered with the teachings contained in the detailed disclosure along with the accompanying drawings.

### DESCRIPTION OF THE INVENTION

### Definitions

The term “negative capillary pressure” refers to a capillary or wicking action that allows for liquid and the like to be pulled into the matrix of the pad away the breast. In one embodiment, the pad when applied to the breast tissue has a negative capillary pressure of about 70 mm of mercury (Hg).

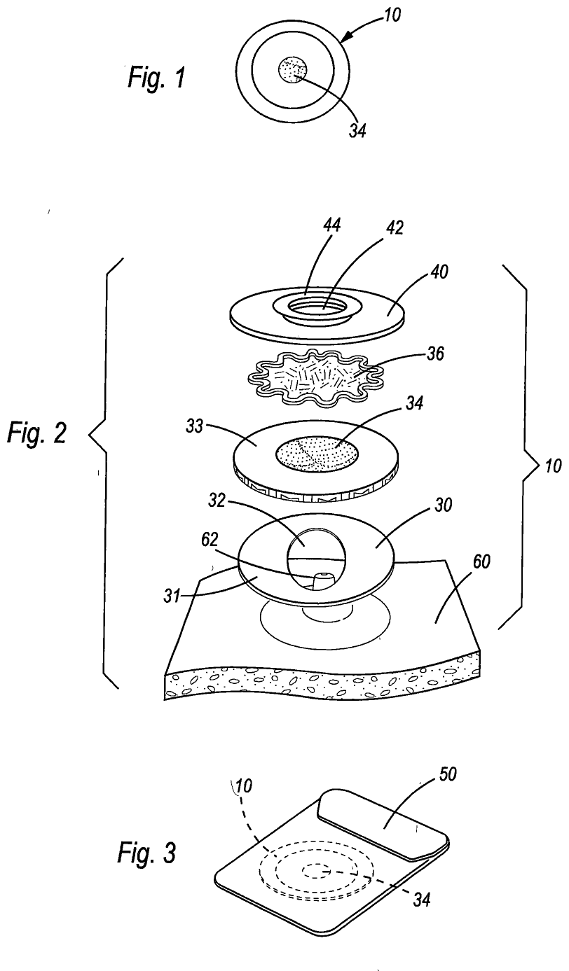

While the preferred polymer used in the breast pad is polyvinyl acetal (PVA), other useable polymers include polyurethane, polyvinyl chloride, polyacrylates, polyester (polyethylene terephthlate), polymethacrylates, polystyrene, polycarbonates and polysulfones, respectively. The PVA foam matrix is dried and cut into circular foam breast pad bodies 10 as shown in FIGS. 1-3. These bodies can be of different selected sizes with top and bottom planar surfaces ranging from a thickness of about 1 mm to about 3 mm to a preferred thickness of about 2 mm. As noted, the reason PVA is used in the preferred embodiment is that the resulting foam sponge pad has a negative capillary pressure (preferably about 70 Hg) which is exerted on the breast and nipple so that breast fluids and the like are pulled from the breast site into the foam matrix of the PVA.

In another embodiment, cellulose paper is used as an alternate material to the PVA sponge breast pad. A suitable cellulose paper is manufactured by Machereg-Nagel Gmbh and sold under the trade name “WATOR”.

The present invention comprises a stacked pad body 10 comprising a base layer 30 having a circular shape with an optional central through going aperture 32. The base layer 30 engages the breast 60 and the aperture 32 is placed over and around the breast nipple 62. The top surface of the base layer 30 is provided with an water proof plastic film cover 31 and the bottom surface is coated with an adhesive layer, covered by a removable backing (not shown) which is preferably clear. A disc 33 which serves as a microfluidic collector pad is mounted on the top surface 31 over the base layer 30. In one embodiment, the collector pad is constructed of a PVA non-adhesive absorbent circular pad 34 which is centered within the soft outer layer 33 of non-absorbent urethane. Preferably specific markers are used from the collected breast fluid which includes both nipple aspirate fluid (NAF) and spontaneous nipple discharge (ND). The specific markers noted below show the NAF collected to be either UP or DOWN in relation to a normal value to determine cancer or pre-cancer. The specific markers used include the following markers:

Prostate Specific Antigens (PSA) which are inversely proportional to disease stage, size of tumor node, and distant metastases, (NAF Indicator Down) (NAF Normal 640 ng/g+_; 62.84+_; 10.28 ug/L) (NAF Cancer 56 ng/g+_0.18+_, 0.03 ugL); Super Oxide Dismutases (SOD—involved in cancer initiation and progression of ROC related damage. ((NAF Indicator Down) (NAF Normal 24.5 mg/g ng/g+_; 62.84+_; 10.28 ug/L) (NAF Cancer 56 mg/g); Ferriten (FTN) which determines proliferation of cancer cells; (NAF Indicator UP) (NAF Normal 55.50+_7.21 ug/L) (NAF Cancer 280.25+_32.32 ug/L)

Additional markers of interest which can be used in connection with the above markers are the following: Plasminogen Activator Inhibitor-1 (PAI-1) which promotes breast cancer invasion and metastasis. (NAF Indicator UP) (NAF Normal 3.4 ng/ml) (NAF Cancer 280.25+32.32 ug/L); Urokinase Plasminogen Activator (uPA) which promotes breast cancer invasion and metastasis. (NAF Indicator UP) (NAF Normal 2.2 ng/ml) (NAF Cancer 2.7 ng/ml) and Thomsen-Friedenreich (TF) predictive for the presence of breast cancer or atypia. (NAF Indicator UP).

A colorimetric circular fluid indicator 36 is formed on a thin film of paper or plastic with a hydrochromic ink coating is mounted on the center pad 34 to indicate to the user that fluid has collected in the center pad 34 from the breast 60/nipple 62. The ink circular indicator 36 is preferably transparent so that the viewer would see a white field in the target area before any NAF is expressed onto the indicator paper. When NAF is translated from the breast fluid onto the paper the indicator chemistry will turn from transparent to blue. If no NAF is deposited the window would remain white/neutral.

The circular indicator can also be a cellulose paper with a color dye such as that marketed as a product for analytical use under the product name—WATOR, manufactured by a German company, Macherey-Nagel GmbH & Co.

A top circular outer seal 40 is mounted over the colorimetric indicator 36 and disc 37. The top circular seal 40 has a round central opening 42 which forms an indicator window 44 which is in turn covered with a clear film coating 46 (not shown). The top circular seal 40 is formed with a soft outer layer of non-absorbent urethane such as that found in the microfluidic pad outer layer.

According to a method of the present invention the diagnostic pad is mounted on the breast for a predetermined time so that the marker carrier layer engages the nipple, and is contact with one or more breast ducts. This allows an extremely small amount of the nipple duct secretion discharged by the nipple duct to be absorbed and immobilized on the absorption layer of the pad.

Various tumor related antigens and markers can be selected with respect to the secretion thus immobilized on the collector pad 34 and tested to determine the amount and presence of same.

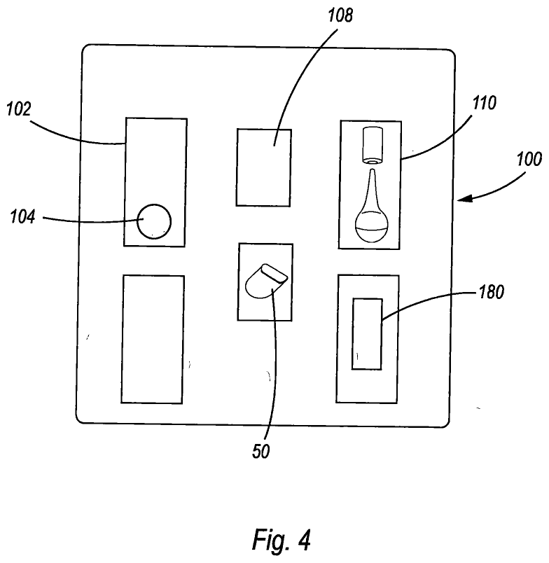

A breast fluid collection kit 100 as seen in FIG. 4 comprising a breast fluid collection pad assembly 102, the breast fluid collection pad assembly 102 being provided with at least one moisture indicator marker 104, which indicates when said adsorbent pad assembly 102 has collected a predetermined amount of moisture from the breast. An adhesive coating can be mounted to the bottom of said adsorbent pad assembly 102 and/or a film cover for the pad if the cover would engage the skin. The adhesive holds the breast assembly on a woman's breast allowing the same to be easily mounted thereon or removed. The collection pad assembly 102 when removed from the breast can be placed in a pouch 50 which is shown in FIG. 3. A breast cleaning pad 108 is mounted in the kit and is used to clean the breast before and/or after breast fluid collection.

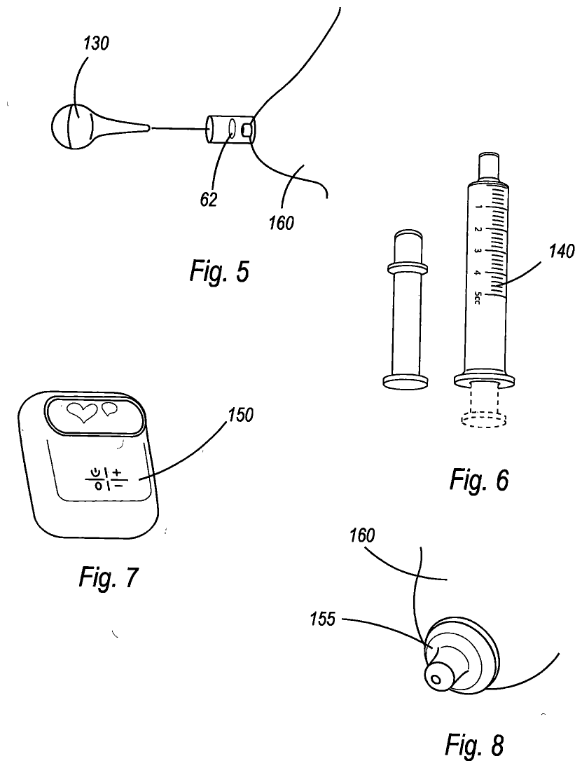

A fluid starter for women who have difficulty in starting or generating breast fluids can additionally be included in the collection kit 100. This fluid starter can include the use of a pump as seen in FIGS. 5-8. One such pump could take the form of a flexible disposable pump and fluid collector 110 is mounted in the kit 100 for application to the nipple area of the breast to start nipple fluid flow if the user has difficulty in starting fluid flow. The pump apparatus in it's simplest form preferably is a suction cup 155 which is placed over the breast nipple and held in place on the breast 160 by adhesive applied inside the suction cup 155 as seen in FIG. 8. The pump apparatus can be utilized directly with the breast or over the stacked body of the pad 10. The manual pump can also take the form of a vacuum squeeze ball 130 as seen in FIG. 5 or as a syringe apparatus 140 as shown in FIG. 6 which applies a vacuum which can be constant or pulsating to the breast. Alternately, a battery powered vacuum pump 150 as seen in FIG. 7 can be used to apply constant pressure and/or pulse mode to the breast. The battery powered pump can also be equipped to provide adjustable psi.

A single silicon nipple cup pump 155 applies vacuum to the breast and nipple by manually inching same forward on the nipple is shown in FIG. 8. Adhesive is used to hold the cup 155 on the breast 160. A collection pad pouch 50 as shown in FIG. 3 is provided with a fluid preservative for holding and shipping the adsorbent pad when the same has collected the breast fluid. The pad is then moisturized and put into a pouch.

It is also envisioned that the kit can contain a small container 180 of Oxytocin, a natural occurring hormone which is used to stimulate the breast and increase breast fluid production if the user has difficulty in naturally producing breast fluid. Oxytocin is naturally produced by the hypothalamus and stored and secreted into the bloodstream from the posterior pituitary gland and can be also be produced synthetically. This container 180 can contain pills with Oxytocin, a nasal inhaler with Oxytocin or contain bottles or containers with fluid or a spray having Oxytocin. If pills are not used or ingested by the patient in the proscribed dosage, the breast and nipple can be covered with Oxytocin fluid or spray and massaged to start the generation of breast fluids for collection purposes. The hormone can be obtained without prescription or with prescription depending upon the usage.

The principles, preferred embodiments and modes of operation of the present invention have been described in the foregoing specification. However, the invention should not be construed as limited to the particular embodiments which have been described above. Instead, the embodiments described here should be regarded as illustrative rather than restrictive. Variations and changes may be made by others without departing from the scope of the present invention as defined by the following claims:

What is claimed is:

1. A breast fluid collection pad assembly for collecting breast fluid discharge and associated biological markers comprising: a pad assembly including a base layer, a fluid collection pad mounted to said base layer, a fluid indicator member mounted on said fluid collection pad and a cover mounted to a fluid indicator member, said cover being provided with an adhesive means allowing it to be mounted to the skin of a patient holding said pad assembly over a breast and said fluid indication means being positioned to show the capture of breast fluids by said fluid collection pad, said fluid indicator member being provided with a target symbol for aligning the center of said fluid indicator member to a breast nipple when mounted to the breast.

2. A breast fluid collection pad assembly of claim 1 wherein said pad is formed with porous polyvinyl acetal foam which provides a negative capillary pressure on said breast.

3. A breast fluid collection pad assembly of claim 1 wherein said fluid indication means changes color when contacted with breast fluid.

4. A breast fluid collection pad assembly of claim 1 wherein said cover is provided with a marking which changes color when contacted by said breast fluid.

5. A breast fluid collection pad assembly of claim 1 wherein said cover is fluid impermeable with a smooth outer surface.

6. A breast fluid collection pad assembly of claim 1 wherein said pad assembly comprises an absorbent pad defines a through going aperture, said pad bottom surface being coated with an adhesive which is covered by a removable backing.

7. A breast fluid collection pad of claim 1 wherein said pad is made of a material which applies a negative capillary pressure of about 70 Hg to the breast when placed on the breast.

8. A breast fluid collection pad of claim 1 wherein said pad is porous nitrocellulose material which can capture said breast fluid.

9. A breast fluid collection pad of claim 1 wherein said pad is a polymeric foam comprised of one or more of a group consisting of polyurethane, polyvinyl chloride, polyacrylates, polyester (polyethylene terephthlate), polymethacrylates, polystyrene, polycarbonates and polysulfones, respectively.

10. A method for making a breast fluid collector assembly to collect nipple fluid for testing to ascertain the presence of cancer or indication of pre-cancer comprising the steps of:
a) removing a pad assembly provided with fluid indicator means from a sterile package and applying it to a breast for a period of time to collect breast fluid;
b) removing said pad assembly with collected breast fluid from the breast;
c) sending said fluid containing pad assembly to a testing facility to determine the presence or absence and quantity of a plurality of specific breast cancer markers in said collected breast fluid, and
d) notifying a physician or specified person of the results of said specific breast cancer marker testing.

11. The method of claim 10 wherein in step c) the specific breast cancer markers found present in said collected breast fluid includes at least two of the following markers: Plasminogen Activation (PAI 1), Urokinase Plasminogen Activator (uPA); and Thomsen-Friedenreich (TF)

12. The method of claim 10 wherein in step c) the specific breast cancer markers found present in said collected breast fluid include at least three or more of the following markers: Prostate Specific Antigens (PSA), Super Oxide Dismutases (SOD-1); Ferriten (FTN); Plasminogen Activation (PAI 1), Urokinase Plasminogen Activator (uPA); and Thomsen-Friedenreich (TF)

13. A breast fluid collection kit comprising a breast fluid collection pad assembly, said breast fluid collection pad assembly comprising a adsorbent pad and a covering mounted to said adsorbent pad, said covering being provided with at least one associated moisture indicator marking which indicates when said adsorbent pad has collected a predetermined amount of fluid from a breast, a breast cleaning pad mounted in said kit, a collection pad pouch provided with a fluid preservative to hold and forward said adsorbent pad when moisturized with breast fluid and a pump device which can be mounted to a breast enclosed in said kit.

14. The breast fluid collection kit as claimed in claim 13 wherein said absorbent pad is formed of a foamed polyvinyl acetal material which applies negative pressure to a breast when mounted thereon.

15. The breast fluid collection kit as claimed in claim 13 wherein said absorbent pad has an adhesive coating bottom surface allowing the adsorbent pad to be mounted on a breast and removed from said breast.

16. The breast fluid collection kit as claimed in claim 13 wherein said kit contains a container with oxytocin hormone.

17. The breast fluid collection kit as claimed in claim 13 wherein said pump device is a syringe.

18. The breast fluid collection kit as claimed in claim 13 wherein said absorbent pad has a covering provided with a marking which changes color when contacted by breast fluid.

19. The breast fluid collection kit as claimed in claim 13 wherein said pump device is a suction cup.

20. The breast fluid collection kit as claimed in claim 13 wherein said pump device is battery driven with contact and pulse modes and has adjustable psi.
